(19) United States
(12) Patent Application Publication
(10) Pub. No.: US 2015/0328188 A1
(43) Pub. Date: Nov. 19, 2015

(54) Combination Therapies for the Treatment of Proliferative Disorders

(71) Applicant: Everardus O. Orlemans, Indianapolis, IN (US)

(72) Inventor: Everardus O. Orlemans, Indianapolis, IN (US)

(21) Appl. No.: 14/716,032

(22) Filed: May 19, 2015

Related U.S. Application Data
(60) Provisional application No. 62/000,271, filed on May 19, 2014.

(51) Int. Cl. A61K 31/416; A61K 45/06

(52) U.S. Cl. CPC A61K 31/416; A61K 45/06

(57) ABSTRACT

Provided is a pharmaceutical composition comprising a combination of an Hsp90 inhibitor and at least one anti-cancer therapeutic. Also provided are methods for treating a proliferative disorder, disease or condition in a subject in need thereof, using a pharmaceutical composition described herein.

### CROSS-REFERENCE TO RELATED APPLICATIONS

This application claims priority to U.S. Provisional Application No. 62/000,271, filed May 19, 2014, the disclosure of which is hereby incorporated by reference in its entirety.

### BACKGROUND OF THE INVENTION

1. Field of the Invention

The invention relates to combination therapies useful in the treatment and/or prevention of diseases and/or conditions related to cell proliferation, such as cancer, inflammation and inflammation-associated disorders, and conditions associated with angiogenesis. Compounds of the invention are also useful in the treatment and/or prevention of infectious diseases, in particular, fungal infections.

2. Description of the Related Art

Cancer is characterized by abnormal cellular proliferation. Cancer cells exhibit a number of properties that make them dangerous to the host, typically including an ability to invade other tissues and to induce capillary ingrowth, which assures that the proliferating cancer cells have an adequate supply of blood. A hallmark of cancerous cells is their abnormal response to control mechanisms that regulate cell division in normal cells and continue to divide until they ultimately kill the host.

Angiogenesis is a highly regulated process under normal conditions, however many diseases are driven by persistent unregulated angiogenesis. Unregulated angiogenesis may either cause a particular disease directly or exacerbate an existing pathological condition. For example, ocular neovascularization has not only been implicated as the most common cause of blindness, but also is believed the dominant cause of many eye diseases. Further, in certain existing conditions, for example arthritis, newly formed capillary blood vessels invade the joints and destroy cartilage, or in the case of diabetes, new capillaries formed in the retina invade the vitreous, bleed, and cause blindness. Growth and metastasis of solid tumors are also dependent on angiogenesis (Folkman, J., Cancer Research, 46, 467-473 (1986), Folkman, J., Journal of the National Cancer Institute, 82, 4-6 (1989). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites such as liver, lung or bone (Weidner, N., et al., The New England Journal of Medicine, 324(1), 1-8 (1991). Under conditions of unregulated angiogenesis, therapeutic methods designed to control, repress, and/or inhibit angiogenesis could lead to the abrogation or mitigation of these conditions and diseases.

Inflammation is related to a variety of disorders such as pain, headaches, fever, arthritis, asthma, bronchitis, menstrual cramps, tendonitis, bursitis, psoriasis, eczema, burns, dermatitis, inflammatory bowel syndrome, Crohn's disease, gastritis, irritable bowel syndrome, ulcerative colitis, vascular diseases, Hodgkin's disease, sclerodoma, rheumatic fever, type I diabetes, myasthenia gravis, sarcoidosis, nephrotic syndrome, Behcet's syndrome, polymyositis, hypersensitivity, conjunctivitis, gingivitis, post-injury swelling, myocardial ischemia, and the like.

Most chemotherapeutic agents act on a specific molecular target thought to be involved in the development of the malignant phenotype. However, a complex network of signaling pathways regulate cell proliferation and the majority of malignant cancers are facilitated by multiple genetic abnormalities in these pathways. Therefore, it is less likely that a therapeutic agent that acts on one molecular target will be fully effective in curing a patient who has cancer.

Heat-shock protein 90 (HSP-90) is a cellular chaperone protein required for the activation of several eukaryotic protein kinases, including the cyclin-dependent kinase CDK4. Geldanamycin, an inhibitor of the protein-refolding activity of HSP-90, has been shown to have anti-proliferative and antitumor activities.

HSP-90 is a molecular chaperone that guides the normal folding, intracellular disposition and proteolytic turnover of many key regulators of cell growth and survival. Its function is subverted during oncogenesis to make malignant transformation possible and to facilitate rapid somatic evolution, and to allow mutant proteins to retain or even gain function. Inhibition of HSP-90 will slow those processes thus has potential therapeutic use (Whitesell L, Lindquist, S L, Nature Rev. Cancer, 2005, 10, 761-72).

Inhibition of HSP-90 is also known to result in up regulation of the expression of the chaperone HSP70. HSP70 up regulation is considered to be of therapeutic benefit for treatment of a wide range of neurodegenerative diseases including, but not limited to: Alzheimer's disease; Parkinson's disease; Dementia with Lewy bodies; Amyotropic lateral sclerosis (ALS); Polyglutamine disease; Huntington's disease; Spinal and bulbar muscular atrophy (SBMA); and Spinocerebellar ataxias (SCA1-3,7). Therefore, the compounds described in the invention are of potential therapeutic use for treatment of such neurodegenerative diseases (Muchowski, P. J., Wacker J. L., Nat. Rev. Neurosci. 2005, 6, 11-22; Shen H. Y., et al. J. Biol. Chem. 2005, 280, 39962-9).

Inhibition of HSP-90 also has anti-fungal activity, both as a standalone therapy and in combination with standard anti-fungal therapies such as the azole class of drugs. Therefore, the compounds described in the invention are of potential therapeutic use for treatment of fungal infections including, but not limited to, life threatening systemic fungal infections (Cowen, L. E., Lindquist, S., Science 2005, 309, 2185-9).

Inhibition of HSP-90 also has antimalarial activity; thus, inhibitors of this protein are useful as antimalarial drugs.

Therefore, there is a continuing need in the art for new methods of treating cancer, inflammation and inflammation-associated disorders, and conditions or diseases related to uncontrolled angiogenesis.

### SUMMARY OF THE INVENTION

In one aspect, the invention encompasses pharmaceutical compositions comprising an Hsp90 inhibitor and at least one anti-cancer therapeutic, and methods employing such compositions in the treatment of diseases and/or conditions related to cell proliferation, such as cancer, inflammation, arthritis, angiogenesis, or the like.

In some embodiments, the composition comprises an Hsp90 inhibitor of formula (I):

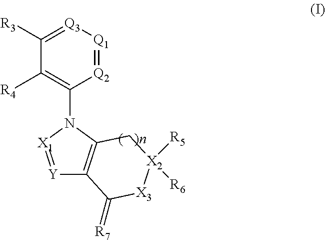

or a pharmaceutically acceptable salt thereof;
at least one anti-cancer therapeutic, or a pharmaceutically acceptable salt thereof; and
at least one pharmaceutically acceptable carrier, solvent, adjuvant or diluent.

The invention further provides methods of treating disease such as cancer, inflammation, arthritis, angiogenesis, and infection in a subject in need thereof, comprising administering to the patient a composition described herein.

The invention also provides the use of a composition described herein for the manufacture of a medicament for use in treating cancer, inflammation, arthritis, angiogenesis, or infection.

The invention also provides methods of treating a disease or condition related to cell proliferation comprising administering a therapeutically effective amount of a composition described herein to a subject in need thereof.

The invention also provides methods of treating a disease or condition related to cell proliferation comprising administering a therapeutically effective amount of a composition described herein to a subject in need thereof, where the disease of condition is cancer, inflammation, angiogenesis, infection or arthritis.

The invention further provides methods of treating a subject suffering from a disease or disorder of proteins that are either client proteins for HSP-90 or indirectly affect its client proteins, comprising administering to a subject in need thereof a therapeutically effective amount of a composition described herein.

The invention further provides methods of treating a subject suffering from a disease or disorder of proteins that are either client proteins for HSP-90 or indirectly affect its client proteins, comprising administering to a subject in need of such treatment a therapeutically effective amount of a composition described herein, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

The invention further provides methods of treating a subject suffering from a fibrogenetic disorder of proteins that are either client proteins for HSP-90 or indirectly affect its client proteins, comprising administering to a subject in need of such treatment a therapeutically effective amount of composition described herein, wherein the fibrogenetic disorder is selected from the group of scleroderma, polymyositis, systemic lupus, rheumatoid arthritis, liver cirrhosis, keloid formation, interstitial nephritis and pulmonary fibrosis.

The invention further provides a composition described herein in a kit with instructions for using the composition.

The invention also provides methods of treating a disease or condition related to cell proliferation comprising administering a therapeutically effective amount of a composition described herein to a subject in need thereof in combination with an anti-cancer therapy.

### DETAILED DESCRIPTION OF THE INVENTION

In a one aspect, the invention encompasses a pharmaceutical composition comprising:

an Hsp90 inhibitor, or a pharmaceutically acceptable salt thereof;
at least one anti-cancer therapeutic, or a pharmaceutically acceptable salt thereof; and
at least one pharmaceutically acceptable carrier, solvent, adjuvant or diluent.

In some embodiments, the Hsp90 inhibitor is a compound of formula (I):

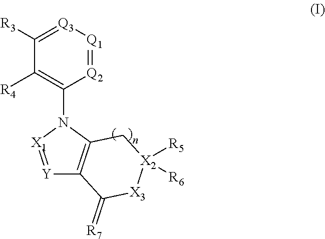

or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
   R₂₂ is
      (i) heteroaryl,
      (ii) aryl,
      (iii) saturated or unsaturated C₃-C₁₀ cycloalkyl, or
      (iv) saturated or unsaturated C₂-C₁₀ heterocycloalkyl, wherein each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
   each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₃, wherein
   Z is OR₀ or —N(R₃₀)₂, wherein
      each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
      R_{O} is —H, —C₁-C₁₀ alkyl, —C₂-C₁₀ alkenyl, —C₂-C₁₀ alkynyl, aryl, heteroaryl, or —C₁-C₆ acyl;
   R₂₃ is
      (1) heteroaryl,
      (2) aryl,
      (3) saturated or unsaturated C₅-C₁₀ cycloalkyl, or
      (4) saturated or unsaturated C₅-C₁₀ heterocycloalkyl, and the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ are optionally substituted with a group R₅₀ where R₅₀ is:

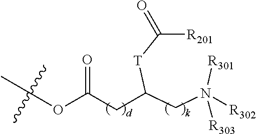

wherein
   d and k are integers independently selected from 1 and 2;
   R₂₀₁ is (C₁-C₆)alkyl where the alkyl is optionally substituted with (C₃-C₇)cycloalkyl, (C₂-C₆) alkenyl, (C₂-C₆)alkynyl, hydroxy, halogen, nitro, or cyano; and
   T is O or NR₂₀₂ where R₂₀₂ is hydrogen or (C₁-C₆)alkyl; and
   R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₁-C₆)alkyl-R₂₂, N—(C₁-C₆ alkenoxy); or N—(C₁-C₆ alkoxy optionally substituted with carboxy);
Y is N or CR_{C}, wherein
   each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
      each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, cyano, nitro, halo(C₁-C₆)alkyl, halo(C₁-C₆)alkoxy, carboxamide, heterocycloalkyl, aryl, or heteroaryl, wherein
         the aryl and heteroaryl groups are optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, or carboxamide;
      R_{C′} is —C₁-C₆ alkyl, —OR_{C″}, or —N(R_{CN})₂, wherein
         R_{C″} is —H, C₁-C₁₀ alkyl, C₁-C₁₀ haloalkyl, C₃-C₇ cycloalkyl, heterocycloalkyl, aryl, or heteroaryl;
      each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
         each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
   each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein
   each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, halo(C₁-C₆)alkoxy, or carboxamide;
   R₂₁ is cyano, —C(O)OH, —C(O)—O(C₁-C₆ alkyl), or a group of the formula

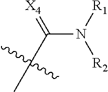

wherein
   R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
      each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
   or R₁ and R₂ together with the nitrogen to which they are both attached, form a heterocycloalkyl which optionally contains one or more additional heteroatoms which are, independently, O, N, S, or N(R_{CN});
   and
   X₄ is O, S, NH, NOH, N—NH₂, N—NHaryl, N—NH—(C₁-C₆ alkyl), or N—(C₁-C₆ alkoxy);
      X₂ and X₃ are independently C, O, N, or S(O)ₚ wherein
p is 0, 1, or 2; and
   n is 0, 1, 2, 3, or 4;
   provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide,
   wherein any two adjacent substituted aryl positions, together with the carbon atoms to which they are attached, form an unsaturated cycloalkyl or heterocycloalkyl; or
   R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is O or S(O)ₚ, then R₆ and R₅ are absent.

In some embodiments, the Hsp90 inhibitor is 4-(6,6-dimethyl-4-oxo-3-trifluoromethyl-4,5,6,7-tetrahydro-indazol-1-yl)-2-(trans-4-hydroxy-cyclohexylamino)-benzamide:

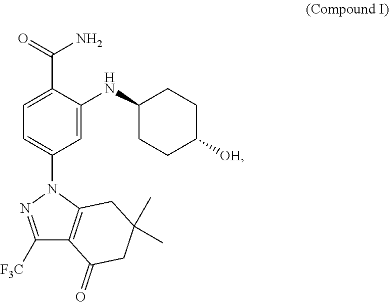 [SMILES: CC1(C)CC(=O)c2c(C(F)(F)F)nn(-c3ccc(C(N)=O)c(N[C@H]4CC[C@H](O)CC4)c3)c2C1]

or a pharmaceutically acceptable salt thereof. Synthesis and characterization data for Compound I is described in U.S. Pat. No. 7,358,370, which is incorporated by reference in its entirety.

In some embodiments, the Hsp90 inhibitor is trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate:

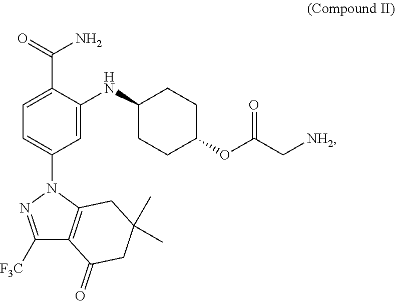 [SMILES: CC1(C)CC(=O)c2c(C(F)(F)F)nn(-c3ccc(C(N)=O)c(N[C@H]4CC[C@H](OC(=O)CN)CC4)c3)c2C1]

or a pharmaceutically acceptable salt thereof. Synthesis and characterization data for Compound II is described in U.S. Pat. No. 7,358,370, which is incorporated by reference in its entirety.

In some embodiments, the Hsp90 inhibitor is 2-(4-(3-acetylcamitineacyloxy)cyclohexy lamino)-4-(1-(3,6,6-trimethyl-4-oxy-4,5,6,7-terahydroindazole))benzamide (2-acetoxy-4-(4-(2-carbamoyl-5-(3,6,6-trimethyl-4-oxo-4,5,6,7-tetrahydro-1H-indazol-1-yl)phenylamino)cyclohexyloxy)-N,N,N-trimethyl-4-oxobutan-1-aminium chloride):

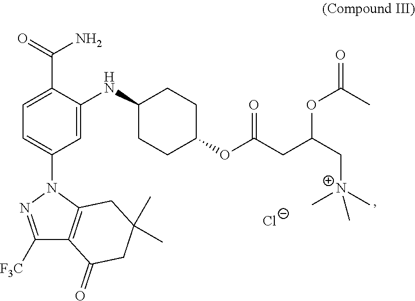 [SMILES: CC(=O)OC(CC(=O)O[C@H]1CC[C@H](Nc2cc(-n3nc(C(F)(F)F)c4c3CC(C)(C)CC4=O)ccc2C(N)=O)CC1)C[N+](C)(C)C ; [Cl-]]

or a pharmaceutically acceptable salt thereof. Synthesis and characterization data for Compound III is described in U.S. Patent Application Publication No. US 2013/0190509, which is incorporated by reference in its entirety.

In some embodiments, the Hsp90 inhibitor is a compound selected from Table 1.

TABLE 1
Hsp90 Inhibitors

2-(allylamino)-4-(3-methyl-4-oxo-4,5,6,7-tetrahydroindol-1-yl)benzamide;
2-(cyclopropylamino)-4-(3-methyl-4-oxo-4,5,6,7-tetrahydroindol-1-yl)benzamide;
2-(2-methoxyethylamino)-4-(3-methyl-4-oxo-4,5,6,7-tetrahydroindol-1-yl)benzamide;
4-(3-methyl-4-oxo-4,5,6,7-tetrahydroindol-1-yl)benzamide;
4-(3,6,6-trimethyl-4-oxo-4,5,6,7-tetrahydroindol-1-yl)benzamide;
4-(3-methyl-4-oxo-4,5,6,7-tetrahydroindol-1-yl)-2-(phenylamino)benzamide;
2-(trans-4-hydroxycyclohexylamino)-4-(3-methyl-4-oxo-4,5,6,7-tetrahydroindol-1-
yl)benzamide;
4-(3-methyl-4-oxo-4,5,6,7-tetrahydroindol-1-yl)-2-(3,4,5-
trimethoxyphenylamino)benzamide;
2-(2-(dimethylamino)ethylamino)-4-(3,6,6-trimethyl-4-oxo-4,5,6,7-tetrahydroindol-1-
yl)benzamide;
2-(2-(dimethylamino)ethylamino)-4-(3-methyl-4-oxo-4,5,6,7-tetrahydroindol-1-
yl)benzamide;
2-(pyridin-4-ylmethylamino)-4-(3,6,6-trimethyl-4-oxo-4,5,6,7-tetrahydroindol-1-
yl)benzamide;
4-(3-methyl-4-oxo-4,5,6,7-tetrahydroindol-1-yl)-2-(pyridin-3-ylmethylamino)benzamide;
tert-butyl 4-(2-carbamoyl-5-(3,6,6-trimethyl-4-oxo-4,5,6,7-tetrahydroindol-1-
yl)phenylamino)piperidine-1-carboxylate;
2-amino-4-(3,6,6-trimethyl-4-oxo-4,5,6,7-tetrahydroindol-1-yl)benzamide;
2-(allylamino)-4-(3,6,6-trimethyl-4-oxo-4,5,6,7-tetrahydroindol-1-yl)benzamide;
2-(1-methylpiperidin-4-ylamino)-4-(3,6,6-trimethyl-4-oxo-4,5,6,7-tetrahydroindol-1-
yl)benzamide;
2-(piperidin-4-ylamino)-4-(3,6,6-trimethyl-4-oxo-4,5,6,7-tetrahydroindol-1-yl)benzamide;
3-butoxy-4-(3,6,6-trimethyl-4-oxo-4,5,6,7-tetrahydroindol-1-yl)benzamide;
2-(2,3-dihydro-1H-inden-1-ylamino)-4-(3,6,6-trimethyl-4-oxo-4,5,6,7-tetrahydroindol-1-
yl)benzamide;
1-(2-carbamoyl-5-(3,6,6-trimethyl-4-oxo-4,5,6,7-tetrahydroindol-1-yl)phenyl)urea
2-Benzylamino-4-(2,6,6-trimethyl-4-oxo-4,5,6,7-tetrahydro-indol-1-yl)-benzamide;
3-Prop-2-ynyloxy-4-(2,6,6-trimethyl-4-oxo-4,5,6,7-tetrahydro-indol-1-yl)-benzamide;
2-Ethynyl-4-(2,6,6-trimethyl-4-oxo-4,5,6,7-tetrahydro-indol-1-yl)-benzamide;
2-(4-Methoxy-phenylamino)-4-(2,6,6-trimethyl-4-oxo-4,5,6,7-tetrahydro-indol-1-yl)-
benzamide;
2-Cyclohexylamino-4-(2,6,6-trimethyl-4-oxo-4,5,6,7-tetrahydro-indol-1-yl)-benzamide;
2-(butylamino)-4-(2,6,6-trimethyl-4-oxo-4,5,6,7-tetrahydroindol-1-yl)benzamide;
4-Methyl-3-(2,6,6-trimethyl-4-oxo-4,5,6,7-tetrahydro-indol-1-yl)-benzamide;
3-(3-thienyl)-4-(2,6,6-trimethyl-4-oxo-4,5,6,7-tetrahydro-1H-indol-1-yl)benzamide;
2-methyl-4-(2,6,6-trimethyl-4-oxo-4,5,6,7-tetrahydro-1H-indol-1-yl)benzamide;
2-[(3-ethynylphenyl)amino]-4-(2,6,6-trimethyl-4-oxo-4,5,6,7-tetrahydro-1H-indol-1-
yl)benzamide;
2-[(4-chlorophenyl)amino]-4-(2,6,6-trimethyl-4-oxo-4,5,6,7-tetrahydro-1H-indol-1-
yl)benzamide;
2-anilino-4-(2-methyl-4-oxo-4,5,6,7-tetrahydro-1H-indol-1-yl)benzamide;
3-anilino-5-(2,6,6-trimethyl-4-oxo-4,5,6,7-tetrahydro-1H-indol-1-yl)pyridine-2-carboxamide;
2-[(3,4,5-trimethoxyphenyl)amino]-4-(2,6,6-trimethyl-4-oxo-4,5,6,7-tetrahydro-1H-indol-1-
yl)benzamide;
2-pyridin-4-yl-4-(2,6,6-trimethyl-4-oxo-4,5,6,7-tetrahydro-1H-indol-1-yl)benzamide;
N-[2-(aminocarbonyl)-5-(2,6,6-trimethyl-4-oxo-4,5,6,7-tetrahydro-1H-indol-1-yl)phenyl]-L-
valine;
2-morpholin-4-yl-4-(2,6,6-trimethyl-4-oxo-4,5,6,7-tetrahydro-1H-indol-1-yl)benzamide;
2-(1H-imidazol-1-yl)-4-(2,6,6-trimethyl-4-oxo-4,5,6,7-tetrahydro-1H-indol-1-yl)benzamide;
4-(3-chloro-2,6,6-trimethyl-4-oxo-4,5,6,7-tetrahydro-1H-indol-1-yl)-2-[(3,4,5-
trimethoxyphenyl)amino]benzamide;
2-[(4-hydroxyphenyl)amino]-4-(2,6,6-trimethyl-4-oxo-4,5,6,7-tetrahydro-1H-indol-1-
yl)benzamide;
2-[(1-ethyl-1H-pyrazol-5-yl)amino]-4-(2,6,6-trimethyl-4-oxo-4,5,6,7-tetrahydro-1H-indol-1-
yl)benzamide;
2-[(5-methylisoxazol-3-yl)amino]-4-(2,6,6-trimethyl-4-oxo-4,5,6,7-tetrahydro-1H-indol-1-
yl)benzamide;
2-{[4-(aminocarbonyl)phenyl]amino}-4-(2-methyl-4-oxo-4,5,6,7-tetrahydro-1H-indol-1-
yl)benzamide;
4-(4-oxo-4,5,6,7-tetrahydro-1H-indol-1-yl)-2-[(3,4,5-trimethoxyphenyl)amino]benzamide;
2-[(6-methoxypyridin-3-yl)amino]-4-(2,6,6-trimethyl-4-oxo-4,5,6,7-tetrahydro-1H-indol-1-
yl)benzamide;
2-(allylamino)-4-(4-oxo-4,5,6,7-tetrahydro-1H-indol-1-yl)benzamide;
4-(2,3-dimethyl-4-oxo-4,5,6,7-tetrahydro-1H-indol-1-yl)benzamide;
3-bromo-4-(2,3-dimethyl-4-oxo-4,5,6,7-tetrahydro-1H-indol-1-yl)benzamide;
2-(allylamino)-4-(2,3-dimethyl-4-oxo-4,5,6,7-tetrahydro-1H-indol-1-yl)benzamide;
4-(2,3-dimethyl-4-oxo-4,5,6,7-tetrahydro-1H-indol-1-yl)-2-[(2-
methoxyethyl)amino]benzamide;
2-({3-[3-(dimethylamino)propoxy]-4-methoxyphenyl}amino)-4-(2,6,6-trimethyl-4-oxo-4,5,6,7-
tetrahydro-1H-indol-1-yl)benzamide;
2-(morpholin-4-ylamino)-4-(2,6,6-trimethyl-4-oxo-4,5,6,7-tetrahydro-1H-indol-1-
yl)benzamide;
2-[(2-methoxyethyl)amino]-4-(3,6,6-trimethyl-4-oxo-4,5,6,7-tetrahydro-1H-indazol-1-
yl)benzamide;
2-[(2-morpholin-4-ylethyl)amino]-4-(3,6,6-trimethyl-4-oxo-4,5,6,7-tetrahydro-1H-indol-1-
yl)benzamide;
2-(pyridin-4-ylamino)-4-(3,6,6-trimethyl-4-oxo-4,5,6,7-tetrahydro-1H-indol-1-yl)benzamide;
2-(acetylamino)-4-(3,6,6-trimethyl-4-oxo-4,5,6,7-tetrahydro-1H-indol-1-yl)benzamide;
2-(4-methylpiperazin-1-yl)-4-(3,6,6-trimethyl-4-oxo-4,5,6,7-tetrahydro-1H-indol-1-
yl)benzamide;
2-[(cyclopropylmethyl)amino]-4-(3,6,6-trimethyl-4-oxo-4,5,6,7-tetrahydro-1H-indazol-1-
yl)benzamide;
2-[(methoxyacetyl)amino]-4-(3,6,6-trimethyl-4-oxo-4,5,6,7-tetrahydro-1H-indol-1-
yl)benzamide;
2-ethyl-4-(3,6,6-trimethyl-4-oxo-4,5,6,7-tetrahydro-1H-indazol-1-yl)benzamide;
2-(butylthio)-4-(3,6,6-trimethyl-4-oxo-4,5,6,7-tetrahydro-1H-indazol-1-yl)benzamide;
2-({3-[2-(dimethylamino)ethoxy]-4-methoxyphenyl}amino)-4-(3,6,6-trimethyl-4-oxo-4,5,6,7-
tetrahydro-1H-indol-1-yl)benzamide;
2-(pyridin-4-ylthio)-4-(3,6,6-trimethyl-4-oxo-4,5,6,7-tetrahydro-1H-indol-1-yl)benzamide;
2-{[(1R)-1-phenylethyl]amino}-4-(3,6,6-trimethyl-4-oxo-4,5,6,7-tetrahydro-1H-indazol-1-
yl)benzamide;
4-[6,6-dimethyl-4-oxo-3-(trifluoromethyl)-4,5,6,7-tetrahydro-1H-indazol-1-yl]-2-[(3,4,5-
trimethoxyphenyl)amino]benzamide;
2-({2-[2-(dimethylamino)ethoxy]pyridin-4-yl}amino)-4-(3,6,6-trimethyl-4-oxo-4,5,6,7-
tetrahydro-1H-indol-1-yl)benzamide;
2-{[1-(N,N-dimethylglycyl)piperidin-4-yl]amino}-4-(3,6,6-trimethyl-4-oxo-4,5,6,7-tetrahydro-
1H-indol-1-yl)benzamide;
4-(6,6-dimethyl-4-oxo-3-phenyl-4,5,6,7-tetrahydro-1H-indol-1-yl)benzamide;
2-[(2-{[2-(aminocarbonyl)-5-(3,6,6-trimethyl-4-oxo-4,5,6,7-tetrahydro-1H-indol-1-
yl)phenyl]amino}ethyl)amino]-2-oxoethyl acetate;
2-{[2-(glycoloylamino)ethyl]amino}-4-(3,6,6-trimethyl-4-oxo-4,5,6,7-tetrahydro-1H-indol-1-
yl)benzamide;
2-{[2-(methylsulfonyl)ethyl]amino}-4-(3,6,6-trimethyl-4-oxo-4,5,6,7-tetrahydro-1H-indazol-1-
yl)benzamide;
2-[(4-methoxyphenyl)amino]-4-(3,6,6-trimethyl-4-oxo-4,5,6,7-tetrahydro-1H-indazol-1-
yl)benzamide;
2-[(6-oxo-1,6-dihydropyridin-3-yl)amino]-4-(3,6,6-trimethyl-4-oxo-4,5,6,7-tetrahydro-1H-
indazol-1-yl)benzamide;
2-(cyclopent-3-en-1-ylamino)-4-[3-(difluoromethyl)-6,6-dimethyl-4-oxo-4,5,6,7-tetrahydro-
1H-indazol-1-yl]benzamide;
2-(cyclobutylamino)-4-(3,6,6-trimethyl-4-oxo-4,5,6,7-tetrahydro-1H-indazol-1-yl)benzamide;
2-[trans-4-(2-Hydroxy-ethoxy)-cyclohexylamino]-4-(3,6,6-trimethyl-4-oxo-4,5,6,7-tetrahydro-
indazol-1-yl)-benzamide;
2-(trans-4-Hydroxy-cyclohexylamino)-4-(3,6,6-trimethyl-4-oxo-4,5,6,7-tetrahydro-indazol-1-
yl)-benzamide;
2-(2-Methoxy-1-methoxymethyl-ethylamino)-4-(3,6,6-trimethyl-4-oxo-4,5,6,7-tetrahydro-
indazol-1-yl)-benzamide;
2-{[3-hydroxy-1-(2-hydroxyethyl)propyl]amino}-4-(3,6,6-trimethyl-4-oxo-4,5,6,7-tetrahydro-
1H-indazol-1-yl)benzamide;
4-[6,6-dimethyl-4-oxo-3-(trifluoromethyl)-4,5,6,7-tetrahydro-1H-indazol-1-yl]-2-{[2-methoxy-
1-(methoxymethyl)ethyl]amino}benzamide;
2-{[3-(methylsulfinyl)phenyl]amino}-4-(3,6,6-trimethyl-4-oxo-4,5,6,7-tetrahydro-1H-indazol-
1-yl)benzamide;
4-[6,6-dimethyl-4-oxo-3-(trifluoromethyl)-4,5,6,7-tetrahydro-1H-indazol-1-yl]-2-{[1-
(methylsulfonyl)piperidin-4-yl]amino}benzamide;
4-(6,6-Dimethyl-4-oxo-3-trifluoromethyl-4,5,6,7-tetrahydro-indazol-1-yl)-2-(trans-4-hydroxy-
cyclohexylamino)-benzamide;
4-(6,6-Dimethyl-4-oxo-3-trifluoromethyl-4,5,6,7-tetrahydro-indazol-1-yl)-2-[trans-4-(2-
hydroxy-ethoxy)-cyclohexylamino]-benzamide;
2-{[1-(3-morpholin-4-ylpropanoyl)piperidin-4-yl]amino}-4-(3,6,6-trimethyl-4-oxo-4,5,6,7-
tetrahydro-1H-indazol-1-yl)benzamide;
2-[trans-4-(2-Amino-ethoxy)-cyclohexylamino]-4-(6,6-dimethyl-4-oxo-3-trifluoromethyl-
4,5,6,7-tetrahydro-indazol-1-yl)-benzamide;
2-[(1-glycylpiperidin-4-yl)amino]-4-(3,6,6-trimethyl-4-oxo-4,5,6,7-tetrahydro-1H-indazol-1-
yl)benzamide;
2-[trans-4-(2-Amino-ethoxy)-cyclohexylamino]-4-(3,6,6-trimethyl-4-oxo-4,5,6,7-tetrahydro-
indazol-1-yl)-benzamide;
2-{[1-(methylsulfonyl)azetidin-3-yl]amino}-4-(3,6,6-trimethyl-4-oxo-4,5,6,7-tetrahydro-1H-
indazol-1-yl)benzamide;
2-{[3-(methylsulfonyl)propyl]amino}-4-(3,6,6-trimethyl-4-oxo-4,5,6,7-tetrahydro-1H-indazol-
1-yl)benzamide;
4-[6,6-dimethyl-4-oxo-3-(trifluoromethyl)-4,5,6,7-tetrahydro-1H-indazol-1-yl]-2-({2-
[(methylsulfonyl)amino]ethyl}amino)benzamide;
4-(3-but-3-en-1-yl-6,6-dimethyl-4-oxo-4,5,6,7-tetrahydro-1H-indazol-1-yl)benzamide;
2-{[(3S)-1-(methylsulfonyl)pyrrolidin-3-yl]amino}-4-(3,6,6-trimethyl-4-oxo-4,5,6,7-tetrahydro-
1H-indazol-1-yl)benzamide;
2-({2-[(dimethylamino)sulfonyl]ethyl}amino)-4-[6,6-dimethyl-4-oxo-3-(trifluoromethyl)-
4,5,6,7-tetrahydro-1H-indazol-1-yl]benzamide;
4-[3-(2-Amino-ethyl)-6,6-dimethyl-4-oxo-4,5,6,7-tetrahydro-indazol-1-yl]-benzamide;
or a pharmaceutically acceptable salt thereof. Synthesis and characterization data of the
above-listed compounds is described in U.S. Pat. No. 7,358,370.

In some embodiments, the composition includes at least one anti-cancer therapeutic, or a pharmaceutically acceptable salt thereof. Suitable anti-cancer therapeutics include, but are not limited to the therapeutics listed in Table 2.

TABLE 2
Anti-cancer Therapeutics.

| Generic Name | IPUAC Name |
|---|---|
| Everolimus | dihydroxy-12-[(2R)-1[(1S,3R,4R)-4-(2-hydroxyethoxy)-3-methoxycyclohexyl]propan-2-yl]-19,30-dimethoxy- 15,17,21,23,29,35-hexamethyl-11,36-dioxa-4-azatricyclo[30.3.1.0 hexatriaconta- 16,24,26,28-tetraene-2,3,10,14,20-pentone 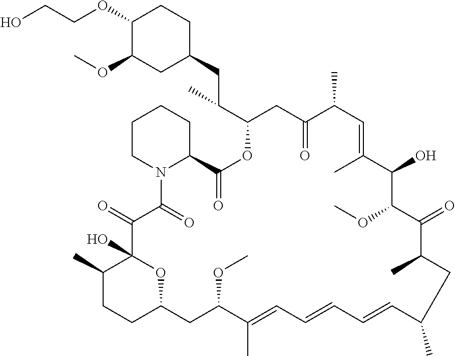 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Erlotinib | N-(3-ethynylphenyl)-6,7-bis(2-methoxyethoxy)quinazolin-4-amine 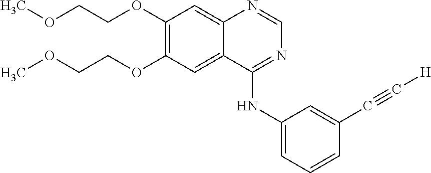 [SMILES: C#Cc1cccc(Nc2ncnc3cc(OCCOC)c(OCCOC)cc23)c1] |
| Abiraterone acetate | [(3S,8R,9S,10R,13S,14S)-10,13-dimethyl-17-pyridin-3-yl-2,3,4,7,8,9,11,12,14,15-decahydro-1H- cyclopenta[a]phenanthren-3-yl]acetate 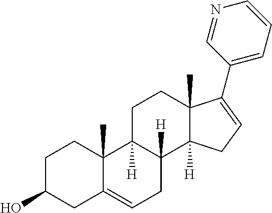 |
| Bleomycin | (3-{[(2′-{(5S,8S,9S,10R,13S)-15-{6-amino-2-[(1S)-3-amino-1{[(2S)-2,3-diamino-3-oxopropyl]amino}-3-oxopropyl]- 5-methylpyrimidin-4-yl}-13-[{[(2R,3S,4S,5S,6S)-3-{[(2R,3S,4S,5R,6R)-4-(carbamoyloxy)-3,5-dihydroxy- 6-(hydroxymethyl)tetrahydro-2H-pyran-2-yl]oxy}-4,5-dihydroxy-6-(hydroxymethyl)tetrahydro-2H- pyran-2-yl]oxy}(1H-imidazol-5-yl)methyl]-9-hydroxy-5-[(1R)-1-hydroxyethyl]-8,10-dimethyl- 4,7,12,15-tetraoxo-3,6,11,14-tetraazapentadec-1-yl}-2,4′-bi-1,3-thiazol- 4-yl)carbonyl]amino}propyl)(dimethyl)sulfonium 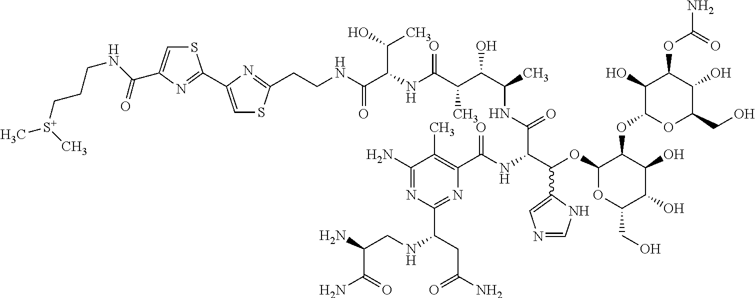 [SMILES: Cc1c(N)nc([C@H](CC(N)=O)NC[C@H](N)C(N)=O)nc1C(=O)N[C@H](C(=O)N[C@H](C)[C@@H](O)[C@H](C)C(=O)N[C@H](C(=O)NCCc1nc(-c2nc(C(=O)NCCC[S+](C)C)cs2)cs1)[C@@H](C)O)C(O[C@@H]1O[C@@H](CO)[C@@H](O)[C@H](O)[C@@H]1O[C@H]1O[C@H](CO)[C@@H](O)[C@H](OC(N)=O)[C@@H]1O)c1cnc[nH]1] |
| Sirolimus | (3S,6R,7E,9R,10R,12R,14S,15E,17E,19E,21S,23S,26R,27R,34aS)-9,10,12,13,14,21,22,23,24,25,26,27,32,33,34,34a- hexadecahydro-9,27-dihydroxy-3-[(1R)-2-[(1S,3R,4R)-4-hydroxy-3-methoxycyclohexyl]-1-methylethyl]-10,21- dimethoxy-6,8,12,14,20,26-hexamethyl-23,27-epoxy-3H-pyrido[2,1-c][1,4]-oxaazacyclohentriacontine- 1,5,11,28,29(4H,6H,31H)-pentone 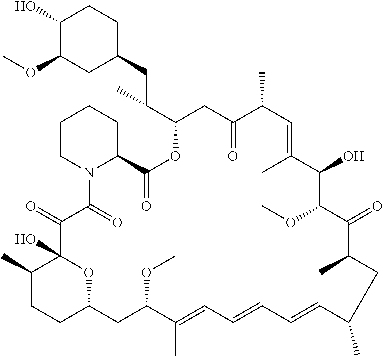 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Arsenic trioxide | 2,4,5-trioxa-1,3-diarsabicyclo[1.1.1]pentane 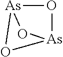 |
| Temsirolimus | (1R,2R,4S)-4-{(2R)-2-[(3S,6R,7E,9R,10R,12R,14S,15E,17E,19E,21S,23S,26R,27R,34aS)-9,27-dihydroxy-10,21- dimethoxy-6,8,12,14,20,26-hexamethyl-1,5,11,28,29-pentaoxo-1,4,5,6,9,10,11,12,13,14,21,22,23,24,25,26,27, 28,29,31,32,33,34,34a-tetracosahydro-3H-23,27-epoxy-pyrido[2,1-c][1,4]oxazacyclohentriacontin-3- yl]propyl}-2-methoxycyclo-hexyl 3-hydroxy-2-(hydroxymethyl)-2-methylpropanoate 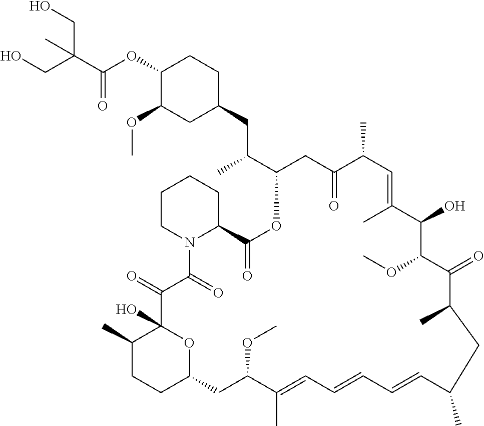 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C] |
| Imatinib | 4-[(4-methylpiperazin-1-yl)methyl]-N-(4-methyl-3-{[4-(pyridin-3-yl)pyrimidin-2-yl]amino}phenyl)benzamide 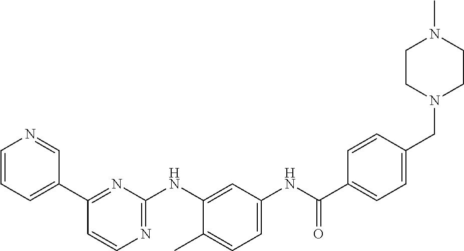 [SMILES: Cc1ccc(NC(=O)c2ccc(CN3CCN(C)CC3)cc2)cc1Nc1nccc(-c2cccnc2)n1] |
| Dasatinib | N-(2-chloro-6-methylphenyl)-2-[[6-[4-(2-hydroxyethyl)-1-piperazinyl]-2-methyl-4-pyrimidinyl]amino]-5- thiazole carboxamide monohydrate 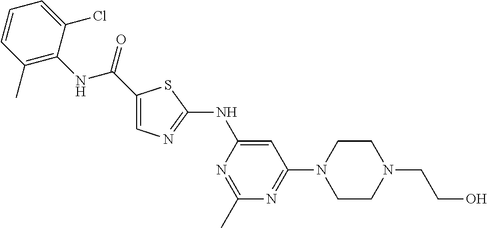 [SMILES: Cc1nc(Nc2ncc(C(=O)Nc3c(C)cccc3Cl)s2)cc(N2CCN(CCO)CC2)n1] |
| Carboplatin | cis-diamine(cyclobutane-1,1-dicarboxylate-O,O′)platinum(II) 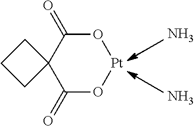 |
| Vandetanib | N-(4-bromo-2-fluorophenyl)-6-methoxy-7-[(1-methylpiperidin-4-yl)methoxy]quinazolin-4-amine 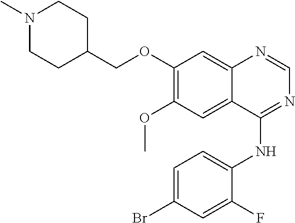 [SMILES: COc1cc2c(Nc3ccc(Br)cc3F)ncnc2cc1OCC1CCN(C)CC1] |
| Lomustine | N-(2-chloroethyl)-N′-cyclohexyl-N-nitrosourea 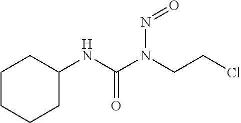 [SMILES: O=NN(CCCl)C(=O)NC1CCCCC1] |
| Cisplatin | (SP-4-2)-diaminedichloroplatinum(II) 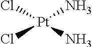 |

In some embodiments, the composition includes an Hsp90 inhibitor selected from Table 1, or a pharmaceutically acceptable salt thereof, and everolimus, erlotinib, abiraterone, bleomycin, sirolimus, arsenic trioxide, temsirolimus, imatinib, dasatinib, carboplatin, vandetanib, lomustine, or cisplatin, or a pharmaceutically acceptable salt thereof.

In some embodiments, the composition includes an Hsp90 inhibitor selected from Table 1, or a pharmaceutically acceptable salt thereof, and everolimus or a pharmaceutically acceptable salt thereof. In other embodiments, the composition includes an Hsp90 inhibitor selected from Table 1, or a pharmaceutically acceptable salt thereof, and bleomycin, or a pharmaceutically acceptable salt thereof. In another embodiment, the composition includes an Hsp90 inhibitor selected from Table 1, or a pharmaceutically acceptable salt thereof, and abiraterone, or a pharmaceutically acceptable salt thereof. In yet another embodiment, the composition includes an Hsp90 inhibitor selected from Table 1, or a pharmaceutically acceptable salt thereof, and erlotinib, or a pharmaceutically acceptable salt thereof.

In some embodiments, the composition includes an Hsp90 of formula (I):

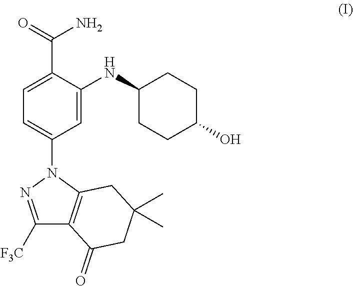 [SMILES: CC1(C)CC(=O)c2c(C(F)(F)F)nn(-c3ccc(C(N)=O)c(N[C@H]4CC[C@H](O)CC4)c3)c2C1]

or a pharmaceutically acceptable salt thereof, and an anti-cancer therapeutic listed in Table 2, or a pharmaceutically acceptable salt thereof.

In some embodiments, the composition includes an Hsp90 of formula (I):

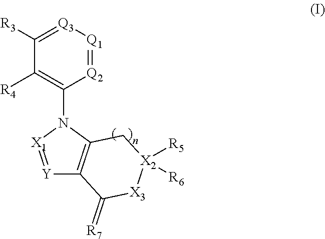

or a pharmaceutically acceptable salt thereof, and everolimus or a pharmaceutically acceptable salt thereof.

In other embodiments, the composition includes an Hsp90 of formula (I):

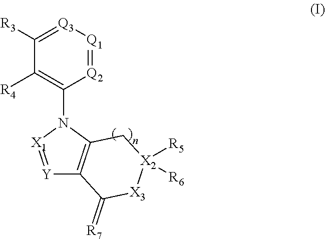

or a pharmaceutically acceptable salt thereof, and abiraterone, or a pharmaceutically acceptable salt thereof.

In other embodiments, the composition includes an Hsp90 of formula (I):

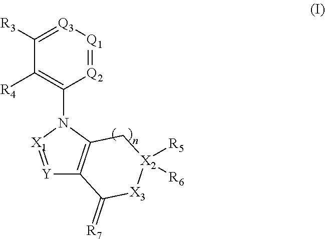

or a pharmaceutically acceptable salt thereof, and bleomycin, or a pharmaceutically acceptable salt thereof.

In other embodiments, the composition includes an Hsp90 of formula (I):

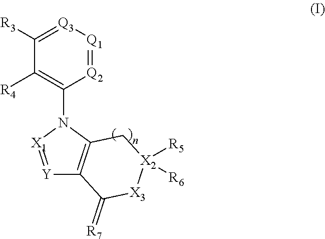

or a pharmaceutically acceptable salt thereof, and erlotinib, or a pharmaceutically acceptable salt thereof.

In some embodiments, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and everolimus or a pharmaceutically acceptable salt thereof.

In some embodiments, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and abiraterone or a pharmaceutically acceptable salt thereof.

In some embodiments, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and bleomycin or a pharmaceutically acceptable salt thereof.

In some embodiments, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and erlotinib or a pharmaceutically acceptable salt thereof.

In some embodiments, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and everolimus or a pharmaceutically acceptable salt thereof.

In some embodiments, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and abiraterone or a pharmaceutically acceptable salt thereof.

In some embodiments, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and bleomycin or a pharmaceutically acceptable salt thereof.

In some embodiments, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and erlotinib or a pharmaceutically acceptable salt thereof.

In another aspect, the invention provides a method for treating a disease or condition related to cell proliferation in a subject in need thereof, the method comprising administering to the subject a composition comprising:

an Hsp90 inhibitor, or a pharmaceutically acceptable salt thereof;
at least one anti-cancer therapeutic, or a pharmaceutically acceptable salt thereof; and
at least one pharmaceutically acceptable carrier, solvent, adjuvant or diluent.

In some embodiments of the method, the disease or condition related to cell proliferation is inflammation, arthritis, or infection. In other embodiments, the condition is angiogenesis. In certain embodiments, the disease is a cancer.

In some embodiments of the method, the Hsp90 inhibitor is a compound listed in Table 1, or a pharmaceutically acceptable salt thereof. In other embodiments of the method, the Hsp90 inhibitor is a compound of formula (I):

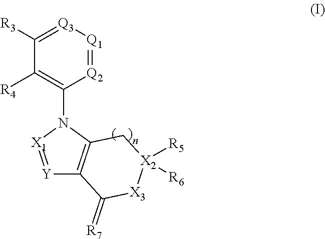

or a pharmaceutically acceptable salt thereof.

In some embodiments of the method, the composition includes an Hsp90 inhibitor listed in Table 1, or a pharmaceutically acceptable salt thereof, and everolimus or a pharmaceutically acceptable salt thereof. In other embodiments of the method, the composition includes an Hsp90 inhibitor listed in Table 1, or a pharmaceutically acceptable salt thereof, and abiraterone, or a pharmaceutically acceptable salt thereof, while in other embodiments of the method the composition includes an Hsp90 inhibitor listed in Table 1, or a pharmaceutically acceptable salt thereof, and bleomycin, or a pharmaceutically acceptable salt thereof. In yet other embodiments of the method, the composition includes an Hsp90 inhibitor listed in Table 1, or a pharmaceutically acceptable salt thereof, and erlotinib, or a pharmaceutically acceptable salt thereof.

In some embodiments of the method, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and everolimus or a pharmaceutically acceptable salt thereof.

In some embodiments of the method, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and abiraterone or a pharmaceutically acceptable salt thereof.

In some embodiments of the method, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and bleomycin or a pharmaceutically acceptable salt thereof.

In some embodiments of the method, the composition includes a compound that is: 4-(6,6-dimethyl-4-oxo-3-trifluoromethyl-4,5,6,7-tetrahydro-indazol-1-yl)-2-(trans-4-hydroxy-cyclohexylamino)-benzamide, or a pharmaceutically acceptable salt thereof, and erlotinib or a pharmaceutically acceptable salt thereof.

In some embodiments of the method, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and everolimus or a pharmaceutically acceptable salt thereof.

In some embodiments of the method, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and abiraterone or a pharmaceutically acceptable salt thereof.

In some embodiments of the method, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and bleomycin or a pharmaceutically acceptable salt thereof.

In some embodiments of the method, the composition includes a compound that is: trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate, or a pharmaceutically acceptable salt thereof, and erlotinib or a pharmaceutically acceptable salt thereof.

In some embodiments of the method, the composition includes a compound that is: 2-(4-(3-acetylcamitineacyloxy)cyclohexylamino)-4-(1-(3,6,6-trimethyl-4-oxy-4,5,6,7-terahydroindazole))benzamide (2-acetoxy-4-(4-(2-carbamoyl-5-(3,6,6-trimethyl-4-oxo-4,5,6,7-tetrahydro-1H-indazol-1-yl)phenylamino)cyclohexyloxy)-N,N,N-trimethyl-4-oxobutan-1-aminium chloride), or a pharmaceutically acceptable salt, and everolimus or a pharmaceutically acceptable salt thereof.

In some embodiments of the method, the composition includes a compound that is: 2-(4-(3-acetylcamitineacyloxy)cyclohexylamino)-4-(1-(3,6,6-trimethyl-4-oxy-4,5,6,7-terahydroindazole))benzamide (2-acetoxy-4-(4-(2-carbamoyl-5-(3,6,6-trimethyl-4-oxo-4,5,6,7-tetrahydro-1H-indazol-1-yl)phenylamino)cyclohexyloxy)-N,N, N-trimethyl-4-oxobutan-1-aminium chloride), or a pharmaceutically acceptable salt thereof, and abiraterone or a pharmaceutically acceptable salt thereof.

In some embodiments of the method, the composition includes a compound that is: 2-(4-(3-acetylcamitineacyloxy)cyclohexylamino)-4-(1-(3,6,6-trimethyl-4-oxy-4,5,6,7-terahydroindazole))benzamide (2-acetoxy-4-(4-(2-carbamoyl-5-(3,6,6-trimethyl-4-oxo-4,5,6,7-tetrahydro-1H-indazol-1-yl)phenylamino)cyclohexyloxy)-N,N, N-trimethyl-4-oxobutan-1-aminium chloride), or a pharmaceutically acceptable salt thereof, and bleomycin or a pharmaceutically acceptable salt thereof.

In some embodiments of the method, the composition includes a compound that is: 2-(4-(3-acetylcamitineacyloxy)cyclohexylamino)-4-(1-(3,6,6-trimethyl-4-oxy-4,5,6,7-terahydroindazole))benzamide (2-acetoxy-4-(4-(2-carbamoyl-5-(3,6,6-trimethyl-4-oxo-4,5,6,7-tetrahydro-1H-indazol-1-yl)phenylamino)cyclohexyloxy)-N,N,N-trimethyl-4-oxobutan-1-aminium chloride), or a pharmaceutically acceptable salt thereof, and erlotinib or a pharmaceutically acceptable salt thereof.

In some embodiments of the method for treating a disease or condition related to cell proliferation in a subject in need thereof, the method comprises administering to the subject:
a composition comprising an Hsp90 inhibitor, or a pharmaceutically acceptable salt thereof, and at least one pharmaceutically acceptable carrier, solvent, adjuvant or diluent; and
a composition comprising at least one anti-cancer therapeutic, or a pharmaceutically acceptable salt thereof, and at least one pharmaceutically acceptable carrier, solvent, adjuvant or diluent.

In some embodiments, the Hsp90 composition and the anti-cancer therapeutic composition are administered separately. Unlike the previously discussed embodiments, wherein the Hsp90 inhibitor and the anti-cancer therapeutic are administered in the same pharmaceutical composition (single composition), the method comprises the administration of the Hsp90 inhibitor and the anti-cancer therapeutic in separate pharmaceutical compositions, each having at least one pharmaceutically acceptable carrier, solvent, adjuvant or diluent (multiple compositions). Administration of compositions can occur concurrently (i.e., at approximately the same time), sequentially (i.e., one after the other), or independently (i.e., with no relation to the other).

Because of the various routes of administration possible for each composition, for some of which delivery is not instantaneous (such as intravascular methods), concurrent administration and sequential administration may both involve some degree of overlap during administration of multiple compositions. Concurrent administration involves the administration of one or more compositions at the same time (i.e., the administration of two or more compositions are initiated at approximately the same time). Sequential administration involves the administration of one composition followed by the administration of a second composition. But sequential administration of a composition with a non-instantaneous delivery, followed by the administration of a second composition, may result in the second composition being administered before the administration of the first composition is complete. So, in some cases, even though two compositions are administered sequentially, their administration can overlap, and therefore may appear to be concurrent.

Independent administration involves the administration of one or more compositions at different times. To differentiate from sequential administration, a second composition administered after a first, non-instantaneously delivered composition is administered independently if the second composition is administered after the administration of the first composition is complete. The time between independently administered compositions can varying from seconds to days.

In another aspect, the invention encompasses the use of a therapeutically effective amount of a composition described herein for the preparation of a medicament for the treatment of cancer, inflammation, or arthritis in a patient in need of such treatment.

In another aspect, the invention encompasses a package comprising a composition described herein in a kit with instructions on how to use the compound.

In another aspect, the invention encompasses the use of a therapeutically effective amount of a composition described herein for the preparation of a medicament for the treatment of a disease or condition related to cell proliferation in a subject in need of such treatment.

In another aspect, the invention encompasses the use of a therapeutically effective amount of a composition described herein for the preparation of a medicament for the treatment of a disease or condition related to cell proliferation in a subject in need of such treatment, wherein the disease or condition is cancer, inflammation, or arthritis.

In another aspect, the invention encompasses the use of a therapeutically effective amount of a composition described herein for the preparation of a medicament for the treatment of a disease or disorder related to the activity of heat shock protein 90, in a subject in need of such.

In another aspect, the invention encompasses the use of therapeutically effective amount of a composition described herein for the preparation of a medicament for the treatment of a disease or disorder related to the activity of heat shock protein 90 and/or its client proteins, in a subject in need of such, wherein the HSP-90 mediated disorder is selected from the group of inflammatory diseases, infections, autoimmune disorders, stroke, ischemia, cardiac disorders, neurological disorders, fibrogenetic disorders, proliferative disorders, tumors, leukemias, neoplasms, cancers, carcinomas, metabolic diseases and malignant disease.

In one embodiment of the method, the treating a disease or condition related to cell proliferation in a subject in need thereof, comprising administering to the subject a composition described herein, reduces the development of multidrug resistant cancerous cells in the subject.

In another preferred aspect, the invention encompasses methods for treating cancer, the methods comprising administration, to a subject in need thereof, of a therapeutically effective amount of a composition described herein, in combination with an anti-cancer therapy. In some embodiments, the anti-cancer therapy is surgery. In some examples, surgery can include, but is not limited to, excision or cryosurgery for the removal of a tumor or cancerous tissue. In other embodiments, the therapy is radio therapy, such as radiation with x-rays or gamma rays.

In some embodiments, the cancer is lung cancer, leukemia, lymphoma, melanoma, ovarian cancer, breast cancer, renal cell carcinoma, neuroendocrine cancer, central nervous system cancer, prostate cancer, colon cancer, head and neck squamous cell carcinoma.

In another aspect, the invention encompasses the use of therapeutically effective amount of a compound or salt of Formula I for the preparation of a medicament for the treatment of a fibrogenetic disorder related to the activity of heat shock protein 90, in a subject in need of such, wherein the fibrogenetic disorder is selected from the group of scleroderma, polymyositis, systemic lupus, rheumatoid arthritis, liver cirrhosis, keloid formation, interstitial nephritis and pulmonary fibrosis.

### Pharmaceutical Compositions

The compositions described herein may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. The pharmaceutical compositions described herein may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compositions disclosed herein may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

The compositions disclosed herein may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The anti-inflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water. Preferred non-human animals include domesticated animals.

The compounds of the present invention may be administered alone or in combination with at least one anti-cancer therapy, e.g., radiation therapy, to a patient in need of such treatment. The additional therapy may be administered concurrently, sequentially or independently of the administration of the compositions disclosed herein.

### Examples

Anti-cancer compounds suitable for combination treatment according to the invention can be selected according to the following criteria: (1) FDA approved, (2) US National Cancer Institute (NCI) 60 human tumor cell line anticancer drug screen (NCI60) growth inhibition (GI50) data available, with more than 2 sensitive cell lines, (3) good quality NCI60 data. On the other hand, drugs with poor quality data, e.g., difference between highest and lowest activity less than 1.5 ¹⁰log units, or lowest activity in multiple cell lines, i.e., insufficient discrimination of lowest activity, making a dose response inconclusive, are unlikely to be suitable. The Hsp90 inhibitors geldanamycin, 17-AAG and 17-DMAG, which have good quality NCI60 data available, are suitable drugs.

For each anti-cancer compound, the current NCI60 growth inhibition (GI50) cell line data (absolute ¹⁰log values for each of the cell lines, except for L-asparaginase, which has both positive and negative ¹⁰log values) are obtained from the Developmental Therapeutics Program (DTP) website (dtp.nci.nih.gov). For each anti-cancer compound, these ¹⁰log values are normalized using a scale from 0 (lowest activity, i.e., no/limited growth inhibition) to 100 (highest activity, i.e., most growth inhibition). These normalized values are correlated to those of Compound I (4-(6,6-dimethyl-4-oxo-3-trifluoromethyl-4,5,6,7-tetrahydro-indazol-1-yl)-2-(trans-4-hydroxy-cyclohexylamino)-benzamide) using a Pearson correlation. Standard deviations based on the entire population (STDEVPA) are also calculated.

Particular combination drugs have a negative Pearson correlation coefficient, (i.e., their predicted sensitivity is anti-correlated to that of Compound I). Examples of particular drugs for use in the invention are listed below in Table 3 together with Pearson analysis results and standard deviations (STDEVPA).

TABLE 3
Example combination drugs

| Rank | Drug | PEARSON | STDEVPA |
|---|---|---|---|
| 1 | Everolimus | −0.30130481 | 32.56501879 |
| 2 | Erlotinib | −0.24856337 | 33.76212789 |
| 3 | Abiraterone | −0.16212469 | 24.69394299 |
| 4 | Bleomycin | −0.15043891 | 30.99192816 |
| 5 | Sirolimus | −0.14103898 | 33.74942757 |
| 6 | Arsenic trioxide | −0.09862521 | 32.22461318 |
| 7 | Temsirolimus | −0.09245061 | 28.12118481 |
| 8 | Imatinib | −0.07952091 | 39.9768978 |
| 9 | Dasatinib | −0.07223224 | 34.59294471 |
| 10 | Carboplatin | −0.06470224 | 34.59581413 |
| 11 | Vandetanib | −0.06458077 | 30.33715129 |
| 12 | Lomustine | −0.01971281 | 34.7619241 |
| 13 | Cisplatin | −0.00706727 | 31.83710892 |

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the scope of the invention as set forth in the claims. To particularly point out and distinctly claim the subject matter regarded as invention, the following claims conclude this specification.

1. A method for treating cancer in a subject in need thereof, the method comprising administering to the subject an Hsp90 inhibitor, or a pharmaceutically acceptable salt thereof, and an anti-cancer therapeutic, or a pharmaceutically acceptable salt thereof.

2. The method of claim 1, wherein the anti-cancer therapeutic is everolimus, erlotinib, abiraterone, bleomycin, sirolimus, arsenic trioxide, temsirolimus, imatinib, dasatinib, carboplatin, vandetanib, lomustine, or cisplatin, or a pharmaceutically acceptable salt thereof.

3-21. (canceled)

22. The method according to claim 1, wherein the cancer is lung cancer, leukemia, lymphoma, melanoma, ovarian cancer, breast cancer, renal cell carcinoma, neuroendocrine cancer, central nervous system cancer, prostate cancer, colon cancer, head and neck squamous cell carcinoma.

23-26. (canceled)

27. The method according to claim 1, further comprising an anti-cancer therapy.

28. The method of claim 27, wherein the anti-cancer therapy is radiation.

29. The method according to claim 1, wherein the HSP90 inhibitor is a compound of the formula I 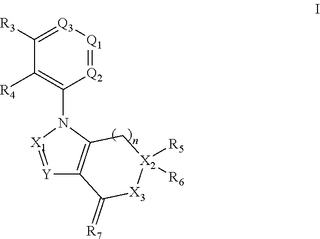
or a pharmaceutically acceptable salt thereof, wherein
R₃ and R₄ are independently
(a) H,
(b) halo, or
(c) a C₁-C₁₅ alkyl group where up to six of the carbon atoms in said alkyl group are optionally replaced independently by R₂₂, carbonyl, ethenyl, ethynyl or a moiety selected from N, O, S, SO₂, or SO, with the proviso that two O atoms, two S atoms, or an O and S atom are not immediately adjacent each other, wherein
R₂₂ is
(i) heteroaryl,
(ii) aryl,
(iii) saturated or unsaturated C₃-C₁₀ cycloalkyl, or
(iv) saturated or unsaturated C₂-C₁₀ heterocycloalkyl, wherein
each aryl, heteroaryl, saturated or unsaturated cycloalkyl, or saturated or unsaturated heterocycloalkyl, independently, is optionally substituted with at least one group, which independently is hydroxy, halo, amino, cyano, carboxy, carboxamido, nitro, oxo, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
each R₂₂ is optionally fused to a C₆-C₁₀ aryl group, C₅-C₈ saturated cyclic group, or a C₅-C₁₀ heterocycloalkyl group;
wherein each (c) moiety is optionally substituted at any available position with C₁-C₁₀ alkyl, C₁-C₁₀ haloalkyl, C₂-C₁₀ alkenyl, C₂-C₁₀ alkynyl, hydroxy, carboxy, carboxamido, oxo, halo, amino, cyano, nitro, —SH, —S—(C₁-C₆)alkyl, —SO₂—(C₁-C₆)alkyl, —SO₂NH₂, —SO₂NH—(C₁-C₆)alkyl, —SO₂NH-aryl, —SO₂-aryl, —SO—(C₁-C₆)alkyl, —SO₂-aryl, C₁-C₆ alkoxy, C₂-C₁₀ alkenyloxy, C₂-C₁₀ alkynyloxy, mono- or di-(C₁-C₁₀)alkylamino, —OC₁-C₁₀ alkyl-Z, or R₂₃, wherein
Z is OR₀ or —N(R₃₀)₂, wherein
each R₃₀ is independently —H or C₁-C₆ alkyl, or N(R₃₀)₂ represents pyrrolidinyl, piperidinyl, piperazinyl, azepanyl, 1,3- or 1,4-diazepanyl, or morpholinyl, each of which is optionally substituted with hydroxy, amino, aminoalkyl, C₁-C₆ alkyl, mono- or di(C₁-C₆)alkylamino, C₁-C₆ alkoxy, or halogen;
R_{O} is —H, —C₁-C₁₀ alkyl, —C₂-C₁₀ alkenyl, —C₂-C₁₀ alkynyl, aryl, heteroaryl, or —C₁-C₆ acyl;
R₂₃ is
(1) heteroaryl,
(2) aryl,
(3) saturated or unsaturated C₅-C₁₀ cycloalkyl, or
(4) saturated or unsaturated C₅-C₁₀ heterocycloalkyl, and
the R₂₃ groups are optionally substituted with at least one group which independently is hydroxy, oxo, halo, amino, cyano, nitro, —SH, —S—(C₁-C₆)alkyl, —SO₂—(C₁-C₆)alkyl, —SO₂-aryl, —SO—(C₁-C₆)alkyl, —SO-aryl, —SO₂NH₂, —SO₂NH—(C₁-C₆)alkyl, —SO₂NH-aryl, (C₁-C₆)alkoxy, or mono- or di-(C₁-C₁₀)alkylamino; and
wherein one or both of R₃ and R₄ are optionally substituted with a group R₅₀ where R₅₀ is: 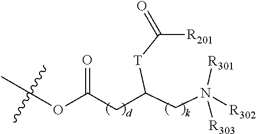
wherein
d and k are integers independently selected from 1 and 2;
R₂₀₁ is (C₁-C₆)alkyl where the alkyl is optionally substituted with (C₃-C₇)cycloalkyl, (C₂-C₆) alkenyl, (C₂-C₆)alkynyl, hydroxy, halogen, nitro, or cyano; and
T is O or NR₂₀₂ where R₂₀₂ is hydrogen or (C₁-C₆)alkyl; and
R₃₀₁ and R₃₀₂ are independently hydrogen or (C₁-C₆)alkyl, and R₃₀₃ is absent, hydrogen, or (C₁-C₆)alkyl;
R₇ is O, S, NH, N—OH, N—NH₂, N—NHR₂₂, N—NH—(C₁-C₆ alkyl), N—O—(C₁-C₆)alkyl-R₂₂, N—(C₁-C₆ alkenoxy); or N—(C₁-C₆ alkoxy optionally substituted with carboxy);
Y is N or CR_{C}, wherein
each R_{C} independently is hydrogen, halogen, cyano, nitro, —C(O)R_{C′}, C₁-C₁₀ alkyl, C₂-C₁₀ alkenyl, C₂-C₁₀ alkynyl, C₁-C₁₀ haloalkyl, C₃-C₇ cycloalkyl, C₃-C₇ cycloalkyl(C₁-C₁₀)alkyl, heterocycloalkyl, aryl, or heteroaryl, wherein
each alkyl, aryl, cycloalkyl, heterocycloalkyl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, cyano, nitro, halo(C₁-C₆)alkyl, halo(C₁-C₆)alkoxy, carboxamide, heterocycloalkyl, aryl, or heteroaryl, wherein
the aryl and heteroaryl groups are optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, or carboxamide;
R_{C′} is —C₁-C₆ alkyl, —OR_{C″}, or —N(R_{CN})₂, wherein
R_{C″} is —H, C₁-C₁₀ alkyl, C₁-C₁₀ haloalkyl, C₃-C₇ cycloalkyl, heterocycloalkyl, aryl, or heteroaryl;
each R_{CN} is independently —H, —C₁-C₁₀ alkyl, —C₁-C₁₀-aloalkyl, —C₃-C₇ cycloalkyl, -heterocycloalkyl, —C₁-C₆ acyl, -aryl, or -heteroaryl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
X₁ is N or CR_{C};
Q₁, Q₂, and Q₃ are independently N or CR_{Q}, wherein one and only one of Q₁, Q₂, and Q₃ is C—R₂₁, and wherein
each R_{Q} is independently hydrogen, halogen, —N(R_{CN})₂, C₁-C₆ alkyl, C₁-C₆ haloalkyl, C₃-C₇ cycloalkyl, aryl, or heteroaryl, or R₂₁, wherein
each alkyl, cycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, halo(C₁-C₆)alkyl, halo(C₁-C₆)alkoxy, or carboxamide;
R₂₁ is cyano, —C(O)OH, —C(O)—O(C₁-C₆ alkyl), or a group of the formula 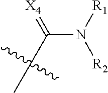
wherein
R₁ and R₂ are independently H, hydroxy, C₁-C₆ alkyl, C₂-C₆ alkenyl, C₂-C₆ alkynyl, heteroaryl, aryl, C₃-C₈ cycloalkyl, heterocycloalkyl, wherein
each alkyl, cycloalkyl, heterocycloalkyl, aryl, and heteroaryl group is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide;
or R₁ and R₂ together with the nitrogen to which they are both attached, form a heterocycloalkyl which optionally contains one or more additional heteroatoms which are, independently, O, N, S, or N(R_{CN});
and
X₄ is O, S, NH, NOH, N—NH₂, N—NHaryl, N—NH—(C₁-C₆ alkyl), or N—(C₁-C₆ alkoxy);
X₂ and X₃ are independently C, O, N, or S(O)ₚ wherein
p is 0, 1, or 2; and
n is 0, 1, 2, 3, or 4;
provided that when
(i) X₂ is C, then
R₅ and R₆ are independently H, C₁-C₆ alkyl, or aryl, wherein the aryl is optionally substituted with from 1-4 groups that are independently C₁-C₆ alkyl, C₁-C₆ alkoxy, halogen, hydroxy, amino, mono- or di-(C₁-C₆) alkylamino, nitro, halo(C₁-C₆)alkyl, halo(C₁-C₆)alkoxy, or carboxamide,
wherein any two adjacent substituted aryl positions, together with the carbon atoms to which they are attached, form an unsaturated cycloalkyl or heterocycloalkyl; or
R₅ and R₆ together with the carbon to which they are attached form a 3-8 membered ring;
(ii) X₂ is N, then R₆ is absent and R₅ is H or C₁-C₆ alkyl;
(iii) X₃ is C, then it is substituted with two groups that are independently H, C₁-C₆ alkyl, or mono- or di-(C₁-C₆)alkylamino(C₁-C₆)alkyl; and
(iv) X₂ is 0 or S(O)ₚ, then R₆ and R₅ are absent.

30. The method according to claim 1, wherein the Hsp90 inhibitor is:
2-(allylamino)-4-(3-methyl-4-oxo-4,5,6,7-tetrahydroindol-1-yl)benzamide;
2-(cyclopropylamino)-4-(3-methyl-4-oxo-4,5,6,7-tetrahydroindol-1-yl)benzamide;
2-(2-methoxyethylamino)-4-(3-methyl-4-oxo-4,5,6,7-tetrahydroindol-1-yl)benzamide;
4-(3-methyl-4-oxo-4,5,6,7-tetrahydroindol-1-yl)benzamide;
4-(3,6,6-trimethyl-4-oxo-4,5,6,7-tetrahydroindol-1-yl)benzamide;
4-(3-methyl-4-oxo-4,5,6,7-tetrahydroindol-1-yl)-2-(phenylamino)benzamide;
2-(trans-4-hydroxycyclohexylamino)-4-(3-methyl-4-oxo-4,5,6,7-tetrahydroindol-1-yl)benzamide;
4-(3-methyl-4-oxo-4,5,6,7-tetrahydroindol-1-yl)-2-(3,4,5-trimethoxyphenylamino)benzamide;
2-(2-(dimethylamino)ethylamino)-4-(3,6,6-trimethyl-4-oxo-4,5,6,7-tetrahydroindol-1-yl)benzamide;
2-(2-(dimethylamino)ethylamino)-4-(3-methyl-4-oxo-4,5,6,7-tetrahydroindol-1-yl)benzamide;
2-(pyridin-4-ylmethylamino)-4-(3,6,6-trimethyl-4-oxo-4,5,6,7-tetrahydroindol-1-yl)benzamide;
4-(3-methyl-4-oxo-4,5,6,7-tetrahydroindol-1-yl)-2-(pyridin-3-ylmethylamino)benzamide;
tert-butyl 4-(2-carbamoyl-5-(3,6,6-trimethyl-4-oxo-4,5,6,7-tetrahydroindol-1-yl)phenylamino)piperidine-1-carboxylate;
2-amino-4-(3,6,6-trimethyl-4-oxo-4,5,6,7-tetrahydroindol-1-yl)benzamide;
2-(allylamino)-4-(3,6,6-trimethyl-4-oxo-4,5,6,7-tetrahydroindol-1-yl)benzamide;
2-(1-methylpiperidin-4-ylamino)-4-(3,6,6-trimethyl-4-oxo-4,5,6,7-tetrahydroindol-1-yl)benzamide;
2-(piperidin-4-ylamino)-4-(3,6,6-trimethyl-4-oxo-4,5,6,7-tetrahydroindol-1-yl)benzamide;
3-butoxy-4-(3,6,6-trimethyl-4-oxo-4,5,6,7-tetrahydroindol-1-yl)benzamide;
2-(2,3-dihydro-1H-inden-1-ylamino)-4-(3,6,6-trimethyl-4-oxo-4,5,6,7-tetrahydroindol-1-yl)benzamide;
1-(2-carbamoyl-5-(3,6,6-trimethyl-4-oxo-4,5,6,7-tetrahydroindol-1-yl)phenyl)urea
2-Benzylamino-4-(2,6,6-trimethyl-4-oxo-4,5,6,7-tetrahydro-indol-1-yl)-benzamide;
3-Prop-2-ynyloxy-4-(2,6,6-trimethyl-4-oxo-4,5,6,7-tetrahydro-indol-1-yl)-benzamide;
2-Ethynyl-4-(2,6,6-trimethyl-4-oxo-4,5,6,7-tetrahydro-indol-1-yl)-benzamide;
2-(4-Methoxy-phenylamino)-4-(2,6,6-trimethyl-4-oxo-4,5,6,7-tetrahydro-indol-1-yl)-benzamide;
2-Cyclohexylamino-4-(2,6,6-trimethyl-4-oxo-4,5,6,7-tetrahydro-indol-1-yl)-benzamide;
2-(butylamino)-4-(2,6,6-trimethyl-4-oxo-4,5,6,7-tetrahydroindol-1-yl)benzamide;
4-Methyl-3-(2,6,6-trimethyl-4-oxo-4,5,6,7-tetrahydro-indol-1-yl)-benzamide;
3-(3-thienyl)-4-(2,6,6-trimethyl-4-oxo-4,5,6,7-tetrahydro-1H-indol-1-yl)benzamide;
2-methyl-4-(2,6,6-trimethyl-4-oxo-4,5,6,7-tetrahydro-1H-indol-1-yl)benzamide;
2-[(3-ethynylphenyl)amino]-4-(2,6,6-trimethyl-4-oxo-4,5,6,7-tetrahydro-1H-indol-1-yl)benzamide;
2-[(4-chlorophenyl)amino]-4-(2,6,6-trimethyl-4-oxo-4,5,6,7-tetrahydro-1H-indol-1-yl)benzamide;
2-anilino-4-(2-methyl-4-oxo-4,5,6,7-tetrahydro-1H-indol-1-yl)benzamide;
3-anilino-5-(2,6,6-trimethyl-4-oxo-4,5,6,7-tetrahydro-1H-indol-1-yl)pyridine-2-carboxamide;
2-[(3,4,5-trimethoxyphenyl)amino]-4-(2,6,6-trimethyl-4-oxo-4,5,6,7-tetrahydro-1H-indol-1-yl)benzamide;
2-pyridin-4-yl-4-(2,6,6-trimethyl-4-oxo-4,5,6,7-tetrahydro-1H-indol-1-yl)benzamide;
N-[2-(aminocarbonyl)-5-(2,6,6-trimethyl-4-oxo-4,5,6,7-tetrahydro-1H-indol-1-yl)phenyl]-L-valine;
2-morpholin-4-yl-4-(2,6,6-trimethyl-4-oxo-4,5,6,7-tetrahydro-1H-indol-1-yl)benzamide;
2-(1H-imidazol-1-yl)-4-(2,6,6-trimethyl-4-oxo-4,5,6,7-tetrahydro-1H-indol-1-yl)benzamide;
4-(3-chloro-2,6,6-trimethyl-4-oxo-4,5,6,7-tetrahydro-1H-indol-1-yl)-2-[(3,4,5-trimethoxyphenyl)amino]benzamide;
2-[(4-hydroxyphenyl)amino]-4-(2,6,6-trimethyl-4-oxo-4,5,6,7-tetrahydro-1H-indol-1-yl)benzamide;
2-[(1-ethyl-1H-pyrazol-5-yl)amino]-4-(2,6,6-trimethyl-4-oxo-4,5,6,7-tetrahydro-1H-indol-1-yl)benzamide;
2-[(5-methyl isoxazol-3-yl)amino]-4-(2,6,6-trimethyl-4-oxo-4,5,6,7-tetrahydro-1H-indol-1-yl)benzamide;
2-{[4-(aminocarbonyl)phenyl]amino}-4-(2-methyl-4-oxo-4,5,6,7-tetrahydro-1H-indol-1-yl)benzamide;
4-(4-oxo-4,5,6,7-tetrahydro-1H-indol-1-yl)-2-[(3,4,5-trimethoxyphenyl)amino]benzamide;
2-[(6-methoxypyridin-3-yl)amino]-4-(2,6,6-trimethyl-4-oxo-4,5,6,7-tetrahydro-1H-indol-1-yl)benzamide;
2-(allylamino)-4-(4-oxo-4,5,6,7-tetrahydro-1H-indol-1-yl)benzamide;
4-(2,3-dimethyl-4-oxo-4,5,6,7-tetrahydro-1H-indol-1-yl)benzamide;
3-bromo-4-(2,3-dimethyl-4-oxo-4,5,6,7-tetrahydro-1H-indol-1-yl)benzamide;
2-(allylamino)-4-(2,3-dimethyl-4-oxo-4,5,6,7-tetrahydro-1H-indol-1-yl)benzamide;
4-(2,3-dimethyl-4-oxo-4,5,6,7-tetrahydro-1H-indol-1-yl)-2-[(2-methoxyethyl)amino]benzamide;
2-({3-[3-(dimethylamino)propoxy]-4-methoxyphenyl}amino)-4-(2,6,6-trimethyl-4-oxo-4,5,6,7-tetrahydro-1H-indol-1-yl)benzamide;
2-(morpholin-4-ylamino)-4-(2,6,6-trimethyl-4-oxo-4,5,6,7-tetrahydro-1H-indol-1-yl)benzamide;
2-[(2-methoxyethyl)amino]-4-(3,6,6-trimethyl-4-oxo-4,5,6,7-tetrahydro-1H-indazol-1-yl)benzamide;
2-[(2-morpholin-4-ylethyl)amino]-4-(3,6,6-trimethyl-4-oxo-4,5,6,7-tetrahydro-1H-indol-1-yl)benzamide;
2-(pyridin-4-ylamino)-4-(3,6,6-trimethyl-4-oxo-4,5,6,7-tetrahydro-1H-indol-1-yl)benzamide;
2-(acetylamino)-4-(3,6,6-trimethyl-4-oxo-4,5,6,7-tetrahydro-1H-indol-1-yl)benzamide;
2-(4-methylpiperazin-1-yl)-4-(3,6,6-trimethyl-4-oxo-4,5,6,7-tetrahydro-1H-indol-1-yl)benzamide;
2-[(cyclopropylmethyl)amino]-4-(3,6,6-trimethyl-4-oxo-4,5,6,7-tetrahydro-1H-indazol-1-yl)benzamide;
2-[(methoxyacetyl)amino]-4-(3,6,6-trimethyl-4-oxo-4,5,6,7-tetrahydro-1H-indol-1-yl)benzamide;
2-ethyl-4-(3,6,6-trimethyl-4-oxo-4,5,6,7-tetrahydro-1H-indazol-1-yl)benzamide;
2-(butylthio)-4-(3,6,6-trimethyl-4-oxo-4,5,6,7-tetrahydro-1H-indazol-1-yl)benzamide;
2-({3-[2-(dimethylamino)ethoxy]-4-methoxyphenyl}amino)-4-(3,6,6-trimethyl-4-oxo-4,5,6,7-tetrahydro-1H-indol-1-yl)benzamide;
2-(pyridin-4-ylthio)-4-(3,6,6-trimethyl-4-oxo-4,5,6,7-tetrahydro-1H-indol-1-yl)benzamide;
2-{[(1R)-1-phenylethyl]amino}-4-(3,6,6-trimethyl-4-oxo-4,5,6,7-tetrahydro-1H-indazol-1-yl)benzamide;
4-[6,6-dimethyl-4-oxo-3-(trifluoromethyl)-4,5,6,7-tetrahydro-1H-indazol-1-yl]-2-[(3,4,5-trimethoxyphenyl)amino]benzamide;
2-({2-[2-(dimethylamino)ethoxy]pyridin-4-yl}amino)-4-(3,6,6-trimethyl-4-oxo-4,5,6,7-tetrahydro-1H-indol-1-yl)benzamide;
2-{[1-(N,N-dimethylglycyl)piperidin-4-yl]amino}-4-(3,6,6-trimethyl-4-oxo-4,5,6,7-tetrahydro-1H-indol-1-yl)benzamide;
4-(6,6-dimethyl-4-oxo-3-phenyl-4,5,6,7-tetrahydro-1H-indol-1-yl)benzamide;
2-[(2-{[2-(aminocarbonyl)-5-(3,6,6-trimethyl-4-oxo-4,5,6,7-tetrahydro-1H-indol-1-yl)phenyl]amino}ethyl)amino]-2-oxoethyl acetate;
2-{[2-(glycoloylamino)ethyl]amino}-4-(3,6,6-trimethyl-4-oxo-4,5,6,7-tetrahydro-1H-indol-1-yl)benzamide;
2-{[2-(methylsulfonyl)ethyl]amino}-4-(3,6,6-trimethyl-4-oxo-4,5,6,7-tetrahydro-1H-indazol-1-yl)benzamide;
2-[(4-methoxyphenyl)amino]-4-(3,6,6-trimethyl-4-oxo-4,5,6,7-tetrahydro-1H-indazol-1-yl)benzamide;
2-[(6-oxo-1,6-dihydropyridin-3-yl)amino]-4-(3,6,6-trimethyl-4-oxo-4,5,6,7-tetrahydro-1H-indazol-1-yl)benzamide;
2-(cyclopent-3-en-1-ylamino)-4-[3-(difluoromethyl)-6,6-dimethyl-4-oxo-4,5,6,7-tetrahydro-1H-indazol-1-yl]benzamide;
2-(cyclobutylamino)-4-(3,6,6-trimethyl-4-oxo-4,5,6,7-tetrahydro-1H-indazol-1-yl)benzamide;
2-[trans-4-(2-Hydroxy-ethoxy)-cyclohexylamino]-4-(3,6,6-trimethyl-4-oxo-4,5,6,7-tetrahydro-indazol-1-yl)-benzamide;
2-(trans-4-Hydroxy-cyclohexylamino)-4-(3,6,6-trimethyl-4-oxo-4,5,6,7-tetrahydro-indazol-1-yl)-benzamide;
2-(2-Methoxy-1-methoxymethyl-ethylamino)-4-(3,6,6-trimethyl-4-oxo-4,5,6,7-tetrahydro-indazol-1-yl)-benzamide;
2-{[3-hydroxy-1-(2-hydroxyethyl)propyl]amino}-4-(3,6,6-trimethyl-4-oxo-4,5,6,7-tetrahydro-1H-indazol-1-yl)benzamide;
4-[6,6-dimethyl-4-oxo-3-(trifluoromethyl)-4,5,6,7-tetrahydro-1H-indazol-1-yl]-2-{[2-methoxy-1-(methoxymethyl)ethyl]amino}benzamide;
2-{[3-(methylsulfinyl)phenyl]amino}-4-(3,6,6-trimethyl-4-oxo-4,5,6,7-tetrahydro-1H-indazol-1-yl)benzamide;
4-[6,6-dimethyl-4-oxo-3-(trifluoromethyl)-4,5,6,7-tetrahydro-1H-indazol-1-yl]-2-{[1-(methylsulfonyl)piperidin-4-yl]amino}benzamide;
4-(6,6-Dimethyl-4-oxo-3-trifluoromethyl-4,5,6,7-tetrahydro-indazol-1-yl)-2-(trans-4-hydroxy-cyclohexylamino)-benzamide;
4-(6,6-Dimethyl-4-oxo-3-trifluoromethyl-4,5,6,7-tetrahydro-indazol-1-yl)-2-[trans-4-(2-hydroxy-ethoxy)-cyclohexylamino]-benzamide;
2-{[1-(3-morpholin-4-ylpropanoyl)piperidin-4-yl]amino}-4-(3,6,6-trimethyl-4-oxo-4,5,6,7-tetrahydro-1H-indazol-1-yl)benzamide;
2-[trans-4-(2-Amino-ethoxy)-cyclohexylamino]-4-(6,6-dimethyl-4-oxo-3-trifluoromethyl-4,5,6,7-tetrahydro-indazol-1-yl)-benzamide;
2-[(1-glycylpiperidin-4-yl)amino]-4-(3,6,6-trimethyl-4-oxo-4,5,6,7-tetrahydro-1H-indazol-1-yl)benzamide;
2-[trans-4-(2-Amino-ethoxy)-cyclohexylamino]-4-(3,6,6-trimethyl-4-oxo-4,5,6,7-tetrahydro-indazol-1-yl)-benzamide;
2-{[1-(methylsulfonyl)azetidin-3-yl]amino}-4-(3,6,6-trimethyl-4-oxo-4,5,6,7-tetrahydro-1H-indazol-1-yl)benzamide;
2-{[3-(methylsulfonyl)propyl]amino}-4-(3,6,6-trimethyl-4-oxo-4,5,6,7-tetrahydro-1H-indazol-1-yl)benzamide;
4-[6,6-dimethyl-4-oxo-3-(trifluoromethyl)-4,5,6,7-tetrahydro-1H-indazol-1-yl]-2-({2-[(methylsulfonyl)amino]ethyl}amino)benzamide;
4-(3-but-3-en-1-yl-6,6-dimethyl-4-oxo-4,5,6,7-tetrahydro-1H-indazol-1-yl)benzamide;
2-{[(3S)-1-(methylsulfonyl)pyrrolidin-3-yl]amino}-4-(3,6,6-trimethyl-4-oxo-4,5,6,7-tetrahydro-1H-indazol-1-yl)benzamide;
2-({2-[(dimethylamino)sulfonyl]ethyl}amino)-4-[6,6-dimethyl-4-oxo-3-(trifluoromethyl)-4,5,6,7-tetrahydro-1H-indazol-1-yl]benzamide; or
4-[3-(2-Amino-ethyl)-6,6-dimethyl-4-oxo-4,5,6,7-tetrahydro-indazol-1-yl]-benzamide;
or a pharmaceutically acceptable salt thereof.

31. The method according to claim 1, wherein the Hsp90 inhibitor is:
4-(6,6-Dimethyl-4-oxo-3-trifluoromethyl-4,5,6,7-tetrahydro-indazol-1-yl)-2-(trans-4-hydroxy-cyclohexylamino)-benzamide,
or a pharmaceutically acceptable salt thereof.

32. The method according to claim 1, wherein the Hsp90 inhibitor is:
trans-4-({2-(aminocarbonyl)-5-[6,6-dimethyl-4-oxo-3-(trifluoromethyl)-4,5,6,7-tetrahydro-1H-indazol-1-yl]phenyl}amino)cyclohexyl glycinate,
or a pharmaceutically acceptable salt thereof.

33. A pharmaceutical composition comprising:
an Hsp90 inhibitor, or a pharmaceutically acceptable salt thereof;
at least one the anti-cancer therapeutic, or a pharmaceutically acceptable salt thereof; and
at least one pharmaceutically acceptable excipient.

34-65. (canceled)
